(19) United States
(12) Patent Application Publication
(10) Pub. No.: US 2022/0202389 A1
(43) Pub. Date: Jun. 30, 2022

(54) METHOD AND APPARATUS FOR DETECTING RESPIRATORY FUNCTION

(71) Applicant: National Tsing Hua University, Hsinchu City (TW)

(72) Inventors: Chi-Shuo Chen, Hsinchu City (TW); Hui-Ling Lin, Hsinchu City (TW); Chaang Ray Chen, Hsinchu City (TW); Rong-Shing Chang, Hsinchu City (TW); Megha Jhunjhunwala, Hsinchu City (TW); Geng-Yue Li, Hsinchu City (TW)

(73) Assignee: National Tsing Hua University, Hsinchu City (TW)

(21) Appl. No.: 17/234,824

(22) Filed: Apr. 20, 2021

(30) Foreign Application Priority Data
Dec. 30, 2020 (TW) 109146891

(51) Int. Cl. A61B 7/00; A61B 5/00

(52) U.S. Cl. CPC A61B 7/003; A61B 5/7267

(57) ABSTRACT

A method and apparatus for detecting a respiratory function are provided. The method includes training a plurality of classification models, receiving a breathing sound by a sound receiver to generate a breathing signal, and classifying the breathing signal by each of the trained classification models to obtain a classification result corresponding to each of the classification models.

### CROSS-REFERENCE TO RELATED APPLICATION

This application claims the priority benefit of Taiwan application serial no. 109146891, filed on Dec. 30, 2020. The entirety of the above-mentioned patent application is hereby incorporated by reference herein and made a part of this specification.

### BACKGROUND

### Technical Field

The disclosure relates to a method and apparatus for detecting a physiological condition, and more particularly to a method and apparatus for detecting a respiratory function.

### Description of Related Art

Due to serious air pollution resulting from rapid development of industry and climate change, respiratory dysfunctions have now become an urgent public health problem. Respiratory diseases can be divided into two categories: restrictive disease and obstructive disease. Interstitial lung disease (ILD) is one of the representatives of restrictive disease, and chronic obstruction pulmonary disease (COPD) is one of the representatives of obstructive disease. COPD often accompanies chronic diseases such as cardiovascular disease, metabolic syndrome, and depression.

Various clinical testing methods have been developed to identify respiratory dysfunctions. Among them, spirometry is a commonly used testing method at present, which is to place a spirometer in the patient's mouth, and ask the patient to inhale hard and then exhale, so as to measure the amount and speed of air in the lungs for evaluation. However, the existing practices all require well-trained experts and complex equipment, thus limiting the popularity to the public.

### SUMMARY

The disclosure provides a method and apparatus for detecting a respiratory function, which can detect the respiratory function in real time to determine whether there is a potential risk of respiratory diseases.

A method for detecting a respiratory function according to the disclosure includes: training a plurality of classification models; collecting a breathing sound by a sound receiver to generate a breathing signal; and respectively classifying the breathing signal by the classification models which have been trained to obtain a classification result corresponding to each of the classification models.

In an embodiment of the disclosure, a step of training the classification models includes: controlling an airflow generator to generate a variety of airflows for breath simulation based on a plurality of parameters in response to a plurality of lung physiological conditions; performing a sound reception on the airflows by the sound receiver and thereby generating a plurality of training signals; and training the classification models by using the training signals.

In an embodiment of the disclosure, a step of training the classification models includes: generating a plurality of training signals based on a plurality of patient breathing patterns, and training the classification models by using the training signals.

In an embodiment of the disclosure, the classification models include a support vector machine (SVM) model, a convolutional neural network (CNN) model, and a compounded CNN with long short term memory (ConvLSTM) model.

In an embodiment of the disclosure, the classification result is one of a mild chronic obstructive lung disease, a severe chronic obstructive lung disease, an interstitial lung disease (ILD), and a normal condition.

In an embodiment of the disclosure, the sound receiver contactlessly receives a sound.

In an embodiment of the disclosure, the sound receiver is a microphone.

An apparatus for detecting a respiratory function according to the disclosure includes: a sound receiver and a computing apparatus. The computing apparatus is coupled to the sound receiver, and configured to: train a plurality of classification models; receive a breathing sound by a sound receiver to generate a breathing signal; and respectively classify the breathing signal by the trained classification models to obtain a classification result corresponding to each of the classification models.

Based on the above, a machine learning algorithm is performed to establish the plurality of classification models, and the breathing sound is directly received to help identify whether the respiratory function is abnormal. Accordingly, the apparatus for detecting the respiratory function can not only identify obstructive diseases from restrictive diseases but also be used as a monitoring device for a patient who requires constant vigilance.

### BRIEF DESCRIPTION OF THE DRAWINGS

FIG. 1 is a block diagram of an apparatus for detecting a respiratory function according to an embodiment of the disclosure.

FIG. 2 is a flowchart of a method for detecting a respiratory function according to an embodiment of the disclosure.

FIG. 3 is a schematic diagram of training classification models according to an embodiment of the disclosure.

FIG. 4 is a comparison diagram of accuracies of classification models according to an embodiment of the disclosure.

### DESCRIPTION OF THE EMBODIMENTS

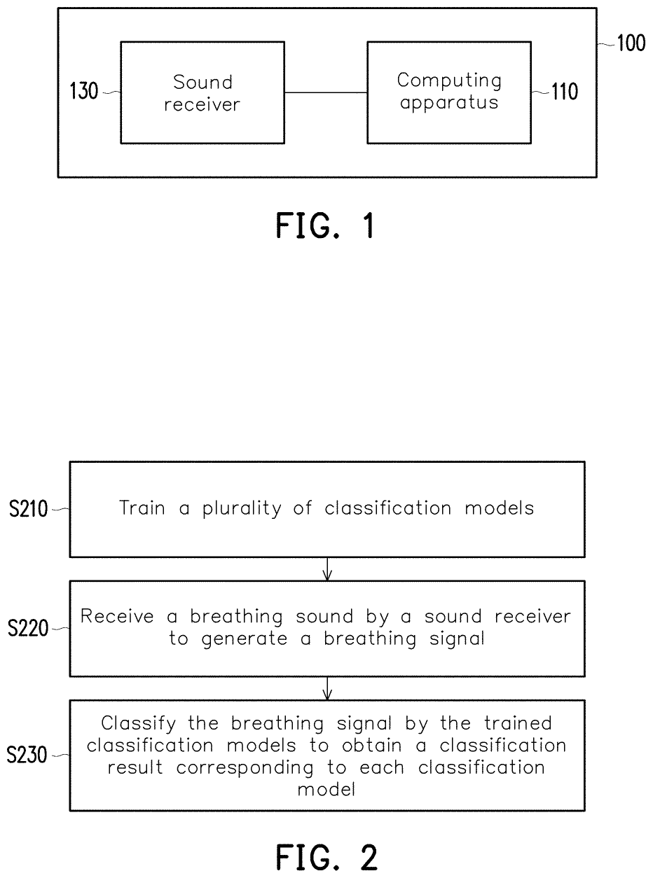

FIG. 1 is a block diagram of an apparatus for detecting a respiratory function according to an embodiment of the disclosure. Referring to FIG. 1, the respiratory function detecting apparatus 100 includes a computing apparatus 110 and a sound receiver 130. The computing apparatus 110 can be coupled to the sound receiver 130 in a wired or wireless manner. The sound receiver 130 can be built in the computing apparatus 110 or externally connected to the computing apparatus 110. The computing apparatus 110 is an electronic apparatus having computing functions. For example, the computing apparatus 110 is a notebook computer, a tablet computer, a smart phone, etc.

The sound receiver 130 is, for example, a handheld microphone, which can contactlessly receive a sound. In an embodiment, the sound receiver 130 and the computing apparatus 110 can be integrated onto a wearable apparatus or a portable electronic apparatus. For example, the sound receiver 130 is disposed on a smart watch or smart phone so that the user receives a breathing sound conveniently.

In an embodiment, in the process of training the classification models, an airflow generator generates a variety of airflows for breath simulation. Specifically, the airflow generator is controlled by the computing apparatus 110 to generate the airflows based on a plurality of parameters in response to a plurality of lung physiological conditions. For example, a pathological condition is simulated according to lung compliance and resistance, thereby reconstructing a variety of breathing patterns to generate the corresponding airflows (the airflows of breath simulation) based on each breathing pattern. A breath airflow depends on the balance between the elastic recoil and airway resistance of the lungs. Lung compliance indicates the change in lung capacity for the transpulmonary pressure, while airway resistance specifies the flow rate under breath pressure. Both compliance and resistance serve as essential indices for quantitatively recapitulating a respiratory system function. The pathological condition is, for example, a chronic obstruction pulmonary disease (COPD), an interstitial lung disease (ILD), and the like.

In the stage of training the models, the sound receiver 130 is moved nearby an output port of the airflow generator, so as to perform a sound reception on the airflows generated by the airflow generator to generate training signals. After that, the training signals are sent to the computing apparatus 110 to train the plurality of classification models.

In addition, in another embodiment, a pneumograph is used to measure a plurality of patients, thereby obtaining a plurality of patient breathing patterns. The pneumograph records the speed and strength of a chest movement during breathing. The computing apparatus 110 generates a plurality of training signals based on the patient breathing patterns, and trains the classification models by using the training signals.

Furthermore, it is also possible to directly receive the breathing sounds of the plurality of patients, thereby establishing a sound database for training.

FIG. 2 is a flowchart of a method for detecting a respiratory function according to an embodiment of the disclosure. Referring to FIG. 1 and FIG. 2 both, first, in step S210, the plurality of classification models are trained. Here, the airflow generator generates a variety of airflows for breath simulation. For example, the computing apparatus 110 controls the airflow generator to generate the airflows based on the parameters in response to a variety of lung physiological conditions. Then, the sound receiver 130 performs a sound reception on the airflows to generate the plurality of training signals. In addition, in another embodiment, the computing apparatus 110 can also generate the plurality of training signals based on the plurality of patient breathing patterns. Here, the audio signals are transformed into training signals by virtue of their power spectral representation using the mel-frequency cepstral coefficients (MFCC). After that, the computing apparatus 110 can train the classification models by the training signals.

In the present embodiment, the classification models include a support vector machine (SVM) model, a convolutional neural network (CNN) model, and a compounded CNN with long short term memory (ConvLSTM) model. However, the examples are only used for description, and the disclosure is not limited thereto.

Here, the SVM model is implemented by using a free software for machine learning in Python programming language (e.g. Scikit-learn library). The Keras library having a TensorFlow backend is used for a neural network.

In the embodiment, a 9-component principal component analysis (PCA) extracts a feature vector for use with the SVM model.

The CNN model includes 2 convolutional layers. The first convolutional layer contains 5 filters. The kernel size of each filter is 5×1, stride is 5, and the filter has a Rectified Linear Unit (ReLU) activation function and L2 regularization. Next, max pooling with dropout layers in sizes 2 and 0.5 is performed. The second convolutional layer contains 20 filters having the same parameters as the first layer's. In a fully connected layer, 30 hidden neurons and a ReLU activation function are used. In a final dense layer, a softmax activation function is used to generate a class-wise classification probability.

In addition, in the ConvLSTM model, an additional 64-unit LSTM layer having a ReLU activation function and L2 regularization is introduced before the dense layers. An optimizer such as Adam is used to perform weight optimization to minimize the categorical cross-entropy loss in both neural network (NN) models.

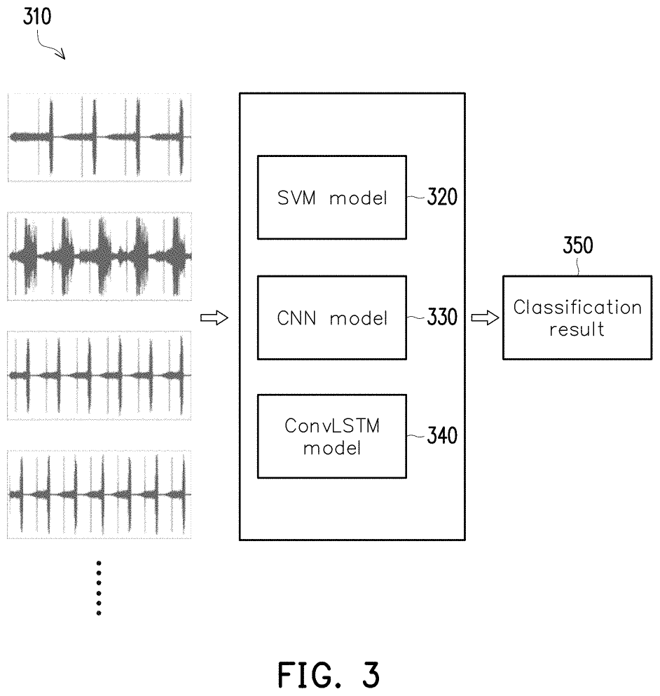

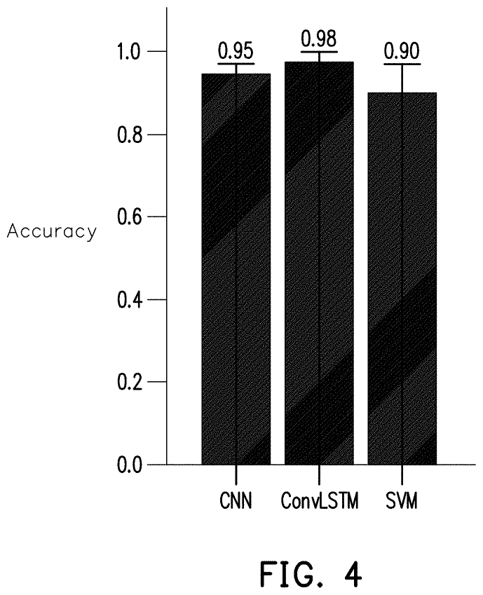

FIG. 3 is a schematic diagram of training classification models according to an embodiment of the disclosure. FIG. 4 is a comparison diagram of accuracies of classification models according to an embodiment of the disclosure. Referring to FIG. 3 and FIG. 4, a plurality of training signals 310 are respectively used as an input of a SVM model 320, a CNN model 330, and a ConvLSTM model 340 to obtain a classification result 350.

In the embodiment, the airflow generator sets the parameters according to specific physiological conditions to establish the plurality of breathing patterns, and then obtains the training signal 310 through the sound receiver 130. For example, two extreme COPD levels (hereafter referred to as mild COPD and severe COPD) are used to identify mild cases from severe cases. 75 different breathing patterns are reconstructed for each disease.

Here, sound waveforms are directly used to train the SVM model 320, the CNN model 330, and the ConvLSTM model 340 to differentiate between a mild COPD, a severe COPD, an ILD, and a normal condition. As shown in FIG. 4, the accuracies of the SVM model 320, the CNN model 330, and the ConvLSTM model 340 are all maintained at a rate above 90%.

In the supervised learning field, the SVM model 320 is a powerful classifier having a high-dimensional feature mapping function, which can separate categories by a hyperplane. However, the shallow machine learning technology is used for small datasets. As the amount of data in a dataset increases and more categories are available to be chosen, overlapping features can overwhelm the support vector, thereby resulting in performance degradation. For a sufficiently large dataset having a large amount of overlapping categories, the CNN model 330 and the ConvLSTM model 340 are superior to the SVM model 320.

After the training of the classification models is completed, in step S220, the breathing sound is received by the sound receiver 130 to generate the breathing signal. Here, the handheld sound receiver contactlessly receives the breathing sound of the target.

After that, in step S230, the breathing signal is respectively classified by the trained classification models (the SVM model 320, CNN model 330, and ConvLSTM model 340 as shown in FIG. 3) to obtain a classification result corresponding to each classification model. Then, cross-validation can be performed according to the plurality of classification results obtained. In addition, in another embodiment, it is possible to use only the classification model with the highest accuracy to perform classification in step S230.

In summary, a machine learning algorithm is performed to establish the plurality of classification models, and the direct reception of breathing sound helps to identify whether the respiratory function is abnormal. Accordingly, the apparatus not only can identify obstructive diseases from restrictive diseases, but also serve as a monitoring device for a patient who requires constant vigilance, and greatly simplify the acquisition of input data.

In contrast to the strict requirement for professional and complex equipment of the existing testing methods, the disclosure achieves the effect of respiratory function monitoring by using a low-cost sound receiver (e.g., a microphone). The method can provide a basis for preliminary diagnosis, and further contribute to the point-of-care testing for respiratory health. Based on the disclosure, a potential respiratory disease can be diagnosed as early as possible, so that a symptom can be alleviated and disease progression can be limited by timely intervention, and a financial burden can also be reduced.

What is claimed is:

1. A method for detecting a respiratory function, comprising:
training a plurality of classification models;
receiving a breathing sound by a sound receiver to generate a breathing signal; and
classifying the breathing signal respectively by the plurality of classification models that have been trained to obtain a classification result corresponding to each of the plurality of classification models.

2. The method for detecting the respiratory function according to claim 1, wherein a step of training the plurality of classification models comprises:
controlling an airflow generator to generate a variety of airflows for breath simulation based on a plurality of parameters in response to a plurality of lung physiological conditions;
performing a sound reception on the airflows by the sound receiver and thereby generating a plurality of training signals; and
training the plurality of classification models by using the plurality of training signals.

3. The method for detecting the respiratory function according to claim 1, wherein a step of training the classification models comprises:
generating a plurality of training signals based on a plurality of patient breathing patterns; and
training the plurality of classification models by using the plurality of training signals.

4. The method for detecting the respiratory function according to claim 1, wherein the plurality of classification models comprise a support vector machine (SVM) model, a convolutional neural network (CNN) model, and a compounded CNN with long short term memory (ConvLSTM) model.

5. The method for detecting the respiratory function according to claim 1, wherein the classification result is one of a mild chronic obstructive lung disease, a severe chronic obstructive lung disease, an interstitial lung disease (ILD), and a normal condition.

6. The method for detecting the respiratory function according to claim 1, wherein the sound receiver contactlessly receives a sound.

7. The method for detecting the respiratory function according to claim 1, wherein the sound receiver is a microphone.

8. An apparatus for detecting a respiratory function, comprising:
a sound receiver; and
a computing apparatus coupled to the sound receiver, and configured to:
train a plurality of classification models;
receive a breathing sound by the sound receiver to generate a breathing signal; and
classify the breathing signal respectively by the plurality of classification models that have been trained to obtain a classification result corresponding to each of the plurality of classification models.

9. The apparatus for detecting the respiratory function according to claim 8, wherein the computing apparatus is coupled to an airflow generator, and configured to control the airflow generator based on a plurality of parameters in response to a plurality of lung physiological conditions to generate a variety of airflows,
the sound receiver performs a sound reception on the airflows and thereby generating a plurality of training signals,
the computing apparatus obtains the plurality of training signals from the sound receiver, and trains the plurality of classification models by using the plurality of training signals.

10. The apparatus for detecting the respiratory function according to claim 8, wherein the computing apparatus is configured to generate the plurality of training signals based on a plurality of patient breathing patterns, and train the plurality of classification models by using the plurality of training signals.

11. The apparatus for detecting the respiratory function according to claim 8, wherein the plurality of classification models comprise a support vector machine (SVM) model, a convolutional neural network (CNN) model, and a compounded CNN with long short term memory (ConvLSTM) model.

12. The apparatus for detecting the respiratory function according to claim 8, wherein the classification result is one of a mild chronic obstructive lung disease, a severe chronic obstructive lung disease, an interstitial lung disease (ILD), and a normal condition.

13. The apparatus for detecting the respiratory function according to claim 8, wherein the sound receiver contactlessly receives a sound.

14. The apparatus for detecting the respiratory function according to claim 8, wherein the sound receiver is a microphone.
